(19) United States
(12) Patent Application Publication
(10) Pub. No.: US 2018/0304252 A1
(43) Pub. Date: Oct. 25, 2018

(54) DETECTION APPARATUS AND INLET STRUCTURE THEREOF

(71) Applicant: SKYLA CORPORATION HSINCHU SCIENCE PARK BRANCH, Hsinchu City (TW)

(72) Inventors: Chia-Chun Wei, Taipei (TW); Hung-Wei Chen, Taipei (TW)

(73) Assignee: SKYLA CORPORATION HSINCHU SCIENCE PARK BRANCH, Hsinchu City (TW)

(21) Appl. No.: 15/622,086

(22) Filed: Jun. 14, 2017

(30) Foreign Application Priority Data
Apr. 19, 2017 (CN) 201710256374.3

(51) Int. Cl. B01L 3/00; B81B 1/00

(52) U.S. Cl. CPC B01L 3/5027; A61B 10/0045; B81B 1/00

(57) ABSTRACT

An inlet structure is adapted to be connected to a microchannel. The inlet structure includes an inlet portion and at least one micro structure. The inlet portion contains an inner surface. The micro structure is disposed in the inlet portion and connected to the inner surface. When a fluid is injected into the inlet portion, the micro structure destroys a surface tension of the fluid to cause the fluid to flow into the microchannel by capillary action.

### CROSS-REFERENCE TO RELATED APPLICATION

This application claims the priority benefit of China application no. 201710256374.3, filed on Apr. 19, 2017. The entirety of the above-mentioned patent application is hereby incorporated by reference herein and made a part of this specification.

### BACKGROUND OF THE INVENTION

### Field of the Invention

The invention is directed to a detection apparatus and an inlet structure thereof and more particularly, to an inlet structure capable of destroying a surface tension of a fluid and a detection apparatus applying the inlet structure.

### Description of Related Art

To satisfy demands of emphasis on preventive medicine, early diagnosis and early treatment in medical treatment, demands on automated testing environment, point of care (POC) or near patient testing and molecular diagnosis have increased. Currently, most of the blood samples are still separated in the centrifuge, and fluids to be tested after the separation are further taken therefrom to a detection apparatus for testing. The detection apparatus may be, for example, a detector instrument or a rapid-screening cassette.

In a conventional detection apparatus, when a fluid is dropped into an inlet of the detection apparatus, the behavior of the fluid is influenced by the surface tension easily and has difficulty of being guided into the inlet. Thus, an absorbent material has to be additionally used to guide and absorb the fluid to allow the fluid to pass through the inlet and enter into the detection apparatus. In this way, production cost of the detection apparatus is increased.

### SUMMARY OF THE INVENTION

The invention provides an inlet structure capable of destroying a surface tension of a fluid to cause the fluid to flow into a microchannel by a capillary action.

The invention provides a detection apparatus capable of allowing a fluid to flow into an inlet structure and a microchannel without the use of any absorbent material, which benefits in cost reduction.

An inlet structure of the invention is adapted to be connected to a microchannel. The inlet structure includes an inlet portion and at least one micro structure. The inlet portion has an inner surface. The micro structure is disposed in the inlet portion and connected with the inner surface. When a fluid is injected into inlet portion, the micro structure destroys the surface tension of the fluid to cause the fluid to flow into the microchannel by a capillary action.

A detection apparatus of the invention includes at least one inlet structure and at least one microchannel. The inlet structure includes an inlet portion and at least one micro structure. The inlet portion has an inner surface. The micro structure is disposed in the inlet portion and connected to the inner surface. The microchannel is connected to the inlet structure. When a fluid is injected into the inlet portion, the micro structure destroys a surface tension of the fluid to cause the fluid to flow into the microchannel by a capillary action.

In an embodiment of the invention, the micro structure is a convex-shaped micro structure protruding from the inner surface of the inlet portion.

In an embodiment of the invention, the micro structure is a concaved-shaped micro structure buried into the inner surface of the inlet portion.

In an embodiment of the invention, when the number of the micro structure is plural, the plurality of micro structures are arranged and spaced equidistantly.

In an embodiment of the invention, the inlet portion further has an inclined surface and a bottom surface. The inner surface is connected between the inclined surface and the bottom surface, and the inner surface is perpendicularly or inclinedly connected to the bottom surface.

In an embodiment of the invention, a side of the micro structure is located at a junction between the inclined surface and the inner surface.

In an embodiment of the invention, a vertical spacing is between a lower surface of the micro structure and the bottom surface of the inlet portion.

In an embodiment of the invention, a lower surface of the micro structure is aligned with the bottom surface of the inlet portion.

In an embodiment of the invention, a top-viewed shape of the inlet portion is circular, and a diameter of the microchannel is less than or equal to a diameter of the inlet portion.

In an embodiment of the invention, a top-viewed shape of the micro structure is polygonal, arcuate or irregular-shaped.

Based on the above, in the inlet structure of the invention, because at least one micro structure is disposed therein, and when the fluid is injected into the inlet portion of the inlet structure, the micro structure can destroy the surface tension of the fluid to cause the fluid to flow into the microchannel by the capillary action. In this way, the inlet structure can guide the fluid to flow inward without the use of any absorbent material. Moreover, the detection apparatus of the invention includes the aforementioned inlet structure which can achieve demands for cost reduction as the use of the absorbent material is not required.

Several exemplary embodiments accompanied with figures are described in detail below to further describe the invention in details.

### BRIEF DESCRIPTION OF THE DRAWINGS

The accompanying drawings are included to provide a further understanding of the present invention, and are incorporated in and constitute a part of this specification. The drawings illustrate embodiments of the present invention and, together with the description, serve to explain the principles of the present invention.

FIG. 1 is a partial top view of a detection apparatus according to an embodiment of the invention.

FIG. 2 is a partial perspective cross-sectional view of the detection apparatus depicted in FIG. 1.

FIG. 3 is a perspective cross-sectional view of an inlet structure according to another embodiment of the invention.

FIG. 4 is a top view of an inlet structure according to yet another embodiment of the invention.

### DESCRIPTION OF THE EMBODIMENTS

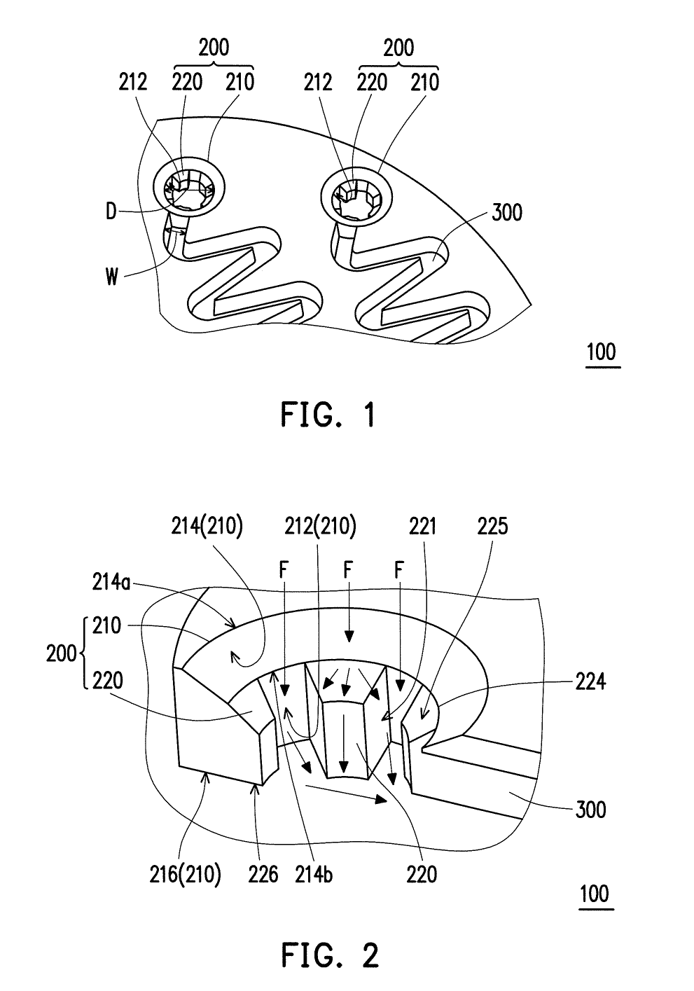

FIG. 1 is a partial top view of a detection apparatus according to an embodiment of the invention. FIG. 2 is a partial perspective cross-sectional view of the detection apparatus depicted in FIG. 1. Referring to both FIG. 1 and FIG. 2, a detection apparatus 100 of the present embodiment includes at least one inlet structure 200 (which only two are schematically illustrated in FIG. 1) and at least one microchannel 300 (which only two are schematically illustrated in FIG. 1). Each of the inlet structures 200 is adapted to be respectively connected to each of the micro channels 300.

To be specific, each of the inlet structures 200 of the detection apparatus 100 of the present embodiment includes an inlet portion 210 and at least one micro structures 220 (which five are schematically illustrated in FIG. 1). The inlet portion 210 has an inner surface 212, and the micro structure 220 is disposed in the inlet portion 210 and connected to the inner surface 212. Particularly, when a fluid F is injected into the inlet portion 210, the micro structure 220 may destroy a surface tension of the fluid F to cause the fluid F to flow into the microchannel 300 by a capillary action.

To be more specific, referring again to FIG. 2, the inlet portion 210 of the present embodiment further has an inclined surface 214, which is inclined downward, and a bottom surface 216. The inner surface 212 is connected between the inclined surface 214 and the bottom surface 216, and the inner surface 212 is perpendicularly connected to the bottom surface 216. The design of the inclined surface 214 is capable of guiding the fluid F to enter into the inner surface 212. However, the inner surface 212 may have an inclined angle (which is also referred to as a draft angle) with respect to the bottom surface 216, but the invention is not limited thereto. As illustrated in FIG. 2, a top-viewed shape of the inlet portion 210 of the present embodiment is embodied as a circle. A diameter of the inlet portion 210 is gradually reduced from a side 214a of the inclined surface 214 which is farthest away from the inner surface 212 toward another side 214b of the inclined surface 214 which is closest to the inner surface 212. In other words, the diameter of the inlet portion 210 of the present embodiment is not a fixed value, but the invention is not limited thereto.

Additionally, in the present embodiment, the plurality of micro structures 220 are arranged and spaced equidistantly, wherein the micro structures 220 are convex-shaped micro structures, namely, the micro structures 220 are disposed on the inner surface 212 in a manner of protruding therefrom. As illustrated in FIG. 2, a side 224 of the micro structure 220 is located at a junction between the inclined surface 214 and the inner surface 212. Nevertheless, the side 224 may be disposed lower than the junction between the inclined surface 214 and the inner surface 212. An upper surface 225 of each micro structure 220 is an inclined surface which is inclined downward. In FIG. 2, an inclined angle of the upper surface 225 is greater than an inclined angle of the inclined surface 214, but the invention is not limited thereto. Each micro structure 220 has three side surfaces 221 (only one of the side surfaces 221 is illustrated in FIG. 2). Two of the side surfaces 221 are connected to the inner surface 212, and the other side surface 221 is connected between the two side surfaces 221. The side surfaces 221 are perpendicularly disposed on the bottom surface 216. However, the side surfaces 221 may be disposed at an inclined angle (which is also referred to as a draft angle) with respect to the bottom surface 216, which is not limited by the invention. A lower surface 226 of each micro structure 220 is aligned with the bottom surface 216 of the inlet portion 210. In other words, a height of the micro structure 220 is substantially the same as a depth of the inner surface 212, but the invention is not limited thereto. In this case, a top-viewed shape of each micro structure 220 is, for example, polygonal, arcuate or irregular-shaped, but the invention is not limited thereto.

As the inlet structure 200 of the present embodiment are disposed with the micro structures 220, when the fluid F is injected into the inlet portion 210 of the inlet structure 200, the micro structures 220 are capable of destroying the surface tension of the fluid F to cause the fluid F to flow to the microchannel 300 connected to the inlet structure 200 by the capillary action. In this way, the inlet structure 200 can guide the fluid F to flow into the inlet structure 200 and the microchannel 300, without the use of any absorbent material. In other words, the detection apparatus 100 of the present embodiment can satisfy the demand for cost reduction.

In addition, the microchannel 300 of the present embodiment are connected to the inlet structure 200, thereby guiding the fluid F to flow into a reaction tank (which is not shown) of the detection apparatus 100 for subsequent analysis. Preferably, a diameter W of the microchannel 300 is less than or equal to a diameter D (which refers to an inner diameter of the inner surface 212 in this case) of the inlet portion 210 to cause the fluid F to flow into the microchannel 300 more easily by the capillary action.

It should be noted that the reference numerals and a part of the contents in the aforementioned embodiment are used in the following embodiments, in which identical reference numerals are adopted to represent identical or similar components, and repeated descriptions of the same technical contents are omitted. For detailed descriptions of the omitted parts, a reference can be found in the aforementioned embodiment, and repeated descriptions thereof are omitted in the following embodiments.

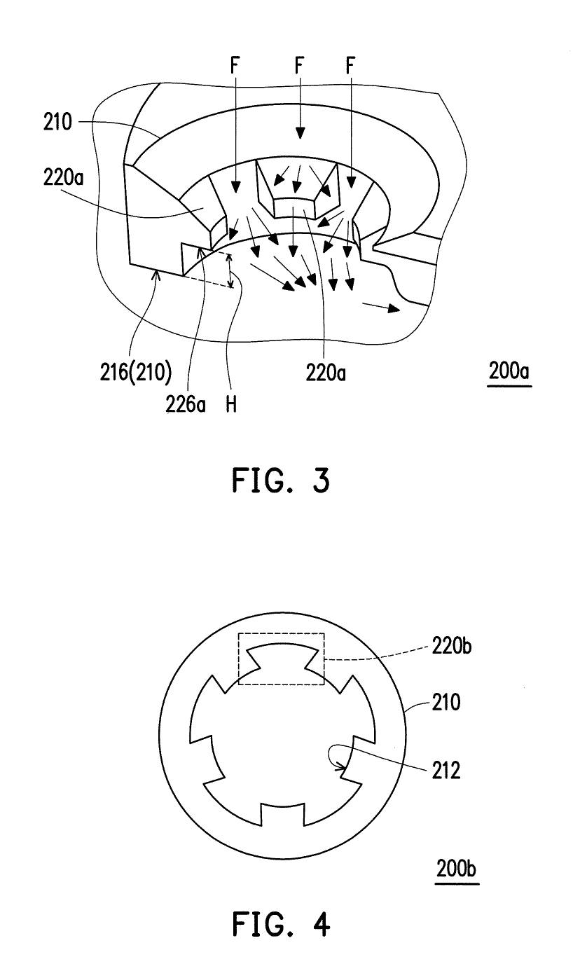

FIG. 3 is a perspective cross-sectional view of an inlet structure according to another embodiment of the invention. Referring to both FIG. 2 and FIG. 3, an inlet structure 200a of the present embodiment is substantially similar to the inlet structure 200 illustrated in FIG. 2, and the difference between the two lies in that there is an vertical spacing H between a lower surface 226a of each micro structure 220a and the bottom surface 216 of the inlet portion 210 in the present embodiment. In other words, the lower surface 226a of each micro structure 220a and the bottom surface 216 of the inlet portion 210 are not on the same plane. When the fluid F is injected into the inlet portion 210 of the inlet structure 200a, the micro structure 220a is capable of destroying the surface tension of the fluid F, and the vertical spacing H between the lower surface 226a of the micro structure 220a and the bottom surface 216 of the inlet portion 210 produces the capillary force more favorably, such that the fluid F can flow into the microchannel 300 connected to the inlet structure 200a more easily by the capillary action.

FIG. 4 is a top view of an inlet structure according to yet another embodiment of the invention. Referring to both FIG. 2 and FIG. 4, an inlet structure 200b of the present embodiment is substantially similar to the inlet structure 200 illustrated in FIG. 2, and the difference between the two lies in that micro structures 220b of the inlet structure 200b of the present embodiment are a plurality of concaved-shaped micro structures, where the micro structures 220b are buried into the inner surface 212 of the inlet portion 210. With the design of angles and sizes of the concavities, the micro structures 220b are capable of destroying the surface tension of the fluid F to cause the fluid F to easily flow into the microchannel 300.

It is to be mentioned that the structural shapes of the micro structures 220, 220a and 220b are not limited by the invention, and it falls within the scope of protection by the invention as long as a non-smooth surface can be defined by the micro structures 220, 220a and 220b and the inner surface 212 of the inlet portion 210. Based on the above design principle, the top-viewed shape of the micro structures 220, 220a and 220b may not only be polygonal, but also arcuate or other irregular-shapes in other embodiments that are not shown.

In view of the foregoing, the detection apparatus of the invention has the inlet structure, and the inlet structure is disposed with the micro structures. Thus, when the fluid is injected into the inlet portion of the inlet structure, the micro structures can destroy the surface tension of the fluid to cause the fluid to flow into the microchannel connected to the inlet structure by the capillary action. In this way, the inlet structure of the invention can guide the fluid to flow into the microchannel without the use of any absorbent material. On the other hand, as the detection apparatus of the invention does not need any absorbent material, the detection apparatus of the invention can satisfy the demand for cost reduction.

It will be apparent to those skilled in the art that various modifications and variations can be made to the structure of the disclosed embodiments without departing from the scope or spirit of the invention. In view of the foregoing, it is intended that the disclosure cover modifications and variations of this specification provided they fall within the scope of the following claims and their equivalents.

What is claimed is:

1. An inlet structure, adapted to be connected to a microchannel, comprising:
an inlet portion, having an inner surface; and
at least one micro structure, disposed in the inlet portion and connected with the inner surface, wherein when a fluid injected into the inlet portion, the micro structure destroys a surface tension of the fluid to cause the fluid to flow into the microchannel by a capillary action.

2. The inlet structure as recited in claim 1, wherein the micro structure is a convex-shaped micro structure protruding from the inner surface of the inlet portion.

3. The inlet structure as recited in claim 1, wherein the micro structure is a concaved-shaped micro structure buried into the inner surface of the inlet portion.

4. The inlet structure as recited in claim 1, wherein when the number of the micro structure is plural, the plurality of micro structures are arranged and spaced equidistantly.

5. The inlet structure as recited in claim 1, wherein the inlet portion further has an inclined surface and a bottom surface, the inner surface is connected between the inclined surface and the bottom surface, and the inner surface is perpendicularly or inclinedly connected to the bottom surface.

6. The inlet structure as recited in claim 5, wherein a side of the micro structure is located at a junction between the inclined surface and the inner surface.

7. The inlet structure as recited in claim 5, wherein a vertical spacing is between a lower surface of the micro structure and the bottom surface of the inlet portion.

8. The inlet structure as recited in claim 5, wherein a lower surface of the micro structure is aligned with the bottom surface of the inlet portion.

9. The inlet structure as recited in claim 1, wherein a top-viewed shape of the inlet portion is circular, and a diameter of the microchannel is less than or equal to a diameter of the inlet portion.

10. The inlet structure as recited in claim 1, wherein a top-viewed shape of the micro structure is polygonal, arcuate or irregular-shaped.

11. A detection apparatus, comprising:
an inlet structure, comprising:
an inlet portion, having an inner surface; and
at least one micro structure, disposed in the inlet portion and connected to the inner surface; and
a microchannel, connected to the inlet structure, wherein when a fluid is injected into the inlet portion of the inlet structure, the micro structure destroys a surface tension of the fluid to cause the fluid to flow into the microchannel by a capillary action.
